(19) United States
(12) Patent Application Publication
(10) Pub. No.: US 2005/0164912 A1
(43) Pub. Date: Jul. 28, 2005

(54) Methods for recovering cleaved peptide from a support after solid phase synthesis

(76) Inventors: Roger D. Bigelow, Boulder, CO (US); Robert O. Cain, Boulder, CO (US)

Correspondence Address: Roche Colorado Corporation, 2075 North 55th Street, Boulder, CO, 80301, US

(21) Appl. No.: 11/022,197

(22) Filed: Dec. 23, 2004

Related U.S. Application Data
(60) Provisional application No. 60/533,655, filed on Dec. 31, 2003.

(51) Int. Cl. C07K 1/02

(52) U.S. Cl. 514/2; 530/333

(57) ABSTRACT

Methods for the solid phase synthesis of peptides and peptide intermediates, in particular methods involving recovering peptides from resin supports at excellent yield. In this invention, an alternating at least partially repeating cycle of shrinking and swelling treatments are used. Each shrinking or swelling part of a cycle may involve one or more washes. The process provides excellent recovery of peptide in a very efficient manner in terms of the number of individual washes and the total volume of wash reagents used.

### PRIORITY CLAIM

The present non-provisional patent Application claims priority under 35 USC § 119(e) from United States Provisional Patent Application having Ser. No. 60/533,655, filed on Dec. 31, 2003, and titled METHODS FOR RECOVERING CLEAVED PEPTIDE FROM A SUPPORT AFTER SOLID PHASE SYNTHESIS, wherein said provisional patent application is commonly owned by the owner of the present patent application and wherein the entire contents of said provisional patent application is incorporated herein by reference.

### FIELD OF THE INVENTION

The invention relates to the solid phase synthesis of peptides. More particularly, the invention relates to the recovery of peptide from solid phase supports using an alternating and at least partially repeating cycle of swelling and shrinking washes.

### BACKGROUND OF THE INVENTION

Many methods for peptide synthesis are described in the literature (for examples, see U.S. Pat. No. 6,015,881; Mergler et al. (1988) Tetrahedron Letters 29: 4005-4008; Mergler et al. (1988) Tetrahedron Letters 29: 4009-4012; Kamber et al. (eds), Peptides, Chemistry and Biology, ESCOM, Leiden (1992) 525-526; Riniker et al. (1993) Tetrahedron Letters 49: 9307-9320; Lloyd-Williams et al. (1993) Tetrahedron Letters 49: 11065-11133; and Andersson et al. (2000) Biopolymers 55: 227-250. The various methods of synthesis are distinguished by the physical state of the phase in which the synthesis takes place, namely liquid phase or solid phase.

In solid phase peptide synthesis (SPPS), an amino acid or peptide group is bound to a solid support resin. Then, successive amino acids or peptide groups are attached to the support-bound peptide until the peptide material of interest is formed. The product of solid-phase synthesis is thus a peptide bound to an insoluble support.

After the peptide is formed, it is usually recovered from the resin. This requires cleaving the attachment between the peptide and resin and thereafter recovering the cleaved peptide using a suitable recovery technique. This is conventionally done by washing the resin one or more times with a reagent into which the cleaved peptide is extracted. Desirably, as much peptide is recovered from the resin as is practical to maximize yield.

Peptides and amino acids from which peptides are synthesized tend to have reactive side groups as well as reactive terminal ends. When synthesizing a peptide, it is important that the amine group on one peptide react with the carboxyl group on another peptide. Undesired reactions at side groups or at the wrong terminal end of a reactant produces undesirable by-products, sometimes in significant quantities. These can seriously impair yield or even ruin the product being synthesized from a practical perspective. To minimize side reactions, it is conventional practice to appropriately mask reactive side groups and terminal ends of reactants to help make sure that the desired reaction occurs.

For example, a typical solid phase synthesis scheme involves attaching a first amino acid or peptide group to the support resin via the carboxyl moiety of the peptide or amino acid. This leaves the amine group of the resin bound material available to couple with additional amino acids or peptide material. Thus, the carboxyl moiety of the additional amino acid or peptide desirably reacts with the free amine group of the resin bound material. To avoid side reactions involving the amine group of the additional amino acid or peptide, such amine group is masked with a protecting group during the coupling reaction. Two well-known amine protecting groups are the BOC group and the FMOC group. Many others also have been described in the literature. After coupling, the protecting group on the N-terminus of the resin bound peptide may be removed, allowing additional amino acids or peptide material to be added to the growing chain in a similar fashion. In the meantime, reactive side chain groups of the amino acid and peptide reactants, including the resin bound peptide material as well as the additional material to be added to the growing chain, typically remain masked with side chain protecting groups throughout the synthesis.

After synthesis, some or all protecting groups can be removed from the peptide product. When at least substantially all of the protecting groups are removed, this is referred to as global deprotection. Global deprotection can occur contemporaneously with cleaving or can be carried out later if the peptide is to be further processed, modified, coupled to additional peptide or other material, etc. Some cleaving reagents not only cleave peptide from the support resin, but also cause global deprotection to occur at the same time. For example, the strongly acidic cleaving reagents associated with BOC chemistry tend to cause global deprotection at the time of cleaving. Other cleaving reagents are more mild than BOC and cleave without causing undue deprotection. The cleaved peptide remains substantially protected after cleaving as a result. The mildly acidic cleaving reagents associated with FMOC chemistry tend to cleave peptides in a globally protected state.

For large-scale production of peptides, issues relating to product recovery and product purity, as well as reagent handling, storage and disposal, can greatly impact the feasibility of the peptide synthesis scheme. Thus, there is a continuing need for peptide synthesis processes capable of efficiently producing peptide materials of commercial interest in large batch quantities with improved yields. Recovery of cleaved peptide from a support resin after solid phase synthesis of the peptide is one aspect of the synthesis in which improvement is needed. Conventional methodologies may tend to leave too much peptide in the resin support.

### SUMMARY OF THE INVENTION

The present invention relates to methods for the solid phase synthesis of peptides and peptide intermediates, in particular methods involving recovering peptides from resin supports at excellent yield. In this invention, an alternating and at least partially repeating cycle of shrinking and swelling treatments are used. Each shrinking or swelling part of a cycle may involve one or more washes. The process provides excellent recovery of peptide in a very efficient manner in terms of the number of individual washes and the total volume of wash reagents used.

A conventional recovery scheme might wash the resin one or more times with DCM and then one or more times with ethanol in order to recover cleaved peptide from the resin. In practical effect, this conventional washing strategy uses an “A-B” strategy, in which the A treatment subjects the resin to one or more swelling washes and the B treatment subjects the resin to one or more shrinking washes. This conventional strategy alternates the shrink and swell treatments, but the A and/or the B treatments are not further repeated in alternating fashion. In contrast, the present invention not only alternates these shrink and swell treatments but also at least partially repeats the cycle of treatments. Thus, if an A-B strategy is followed, the invention would follow up with at least an additional A treatment. If a B-A strategy is followed, the invention would follow up with at least an additional B treatment. For instance, representative embodiments of the invention may use an alternating and at least partially repeating pattern such as one or more of A-B-A; A-B-A-B; A-(B-A)ₙ-Bₘ where n is 2 or more, preferably 2 to 8, and m is 0 or 1; B-A-B; B-A-B-A; B-(A-B)ₙ-Aₘ where n is 2 or more, preferably 2 to 8 and m is 0 or 1; and/or the like.

For instance, an embodiment of the invention using a DCM-alcohol-DCM-alcohol treatment strategy would allow an extra 6 to 10% of peptide to be recovered from the resin as compared to a mere DCM-alcohol treatment strategy, even when the latter DCM-alcohol strategy may have used not only a greater number of individual DCM and alcohol washes but a greater total volume of DCM and alcohol as well. While not wishing to be bound by a particular theory, it is believed that the shrinking treatment of the invention mechanically squeezes or extrudes additional peptide from the resin, allowing additional peptide to be recovered from not just the shrinking wash but also more easily from subsequent swelling washes. In short, the present invention allows higher peptide recovery; and it may accomplish this using lesser volumes of washing reagents in a reduced number of individual washes.

Another conventional recovery scheme might wash the resin to recover peptide in an alternating and at least partially repeating pattern, but does so in the context of BOC chemistry accompanied by practically simultaneous global deprotection. See, e.g., U.S. Pat. No. 4,594,329, which suggests an alternating treatment of chloroform and an ether after cleaving and globally de-protecting BOC-protected peptide from the resin. In contrast, and preferably in the context of using FMOC or FMOC-like chemistry, the present invention finds utility in peptide synthesis where the peptide is cleaved in a protected state, preferably at least substantially globally protected. Using the alternating and at least partially repeating wash strategy in the context of such chemistry provides improved yield as compared to using an otherwise identical wash strategy in the context of cleaving and globally de-protecting chemistry. When a peptide is both cleaved and globally de-protected, the resultant wash will tend to contain a substantial amount of impurities. As a consequence, one would not want to try and recover peptide from the ether washes used in U.S. Pat. No. 4,594,329, for example. Mostly, these washes are likely to contain residues released by deprotection of amino acids. Peptide in those fractions is lost, as a practical matter.

In contrast, using a cleaving chemistry, such as FMOC chemistry, allows peptide to be cleaved without undue deprotection occurring. As a practical matter, the cleaved peptide remains globally protected, and N-terminus protecting group, such as FMOC, remains in place. The reaction is much cleaner, allowing peptide to be recovered practically from both the swelling and shrinking washes, enhancing yield.

### DETAILED DESCRIPTION

The embodiments of the present invention described below are not intended to be exhaustive or to limit the invention to the precise forms disclosed in the following detailed description. Rather, the embodiments are chosen and described so that others skilled in the art can appreciate and understand the principles and practices of the present invention.

The present invention provides a process for recovering peptide material from a support resin with improved yield and efficiency after solid phase synthesis of the peptide of interest. As an overview, peptide initially is cleaved, if not already cleaved, from its support after solid phase peptide synthesis. The peptide is cleaved in a protected state. That is, the peptide is cleaved without undue deprotection of side chain and N-terminus (or carboxy-terminus, as the case may be) protecting groups. FMOC or FMOC-like chemistry is highly preferred for solid phase peptide synthesis, inasmuch as cleaving the resultant peptide in a protected state is relatively straightforward to carry out using mildly acidic cleaving agents. This kind of cleaving reaction is relatively clean in terms of resultant by-products, impurities, etc., making it technically and economically feasible to recover peptide on a large scale basis from both the swelling and shrinking washes, enhancing yield. As used herein, “large scale” with respect to peptide synthesis generally includes the synthesis of peptides in the range of at least 500 g, more preferably at least 2 kg per batch. Large-scale synthesis is typically performed in large reaction vessels, such as steel reaction vessels, that can accommodate quantities of reagents such as resins, solvents, amino acids, chemicals for coupling, and deprotection reactions, that are sized to allow for production of peptides in the kilograms to metric tonnes range.

After cleaving, the support is washed one or more times with a swelling reagent to extract cleaved peptide into the resultant wash(es), and the wash(es) are collected to allow recovery of the peptide from those washes. Note, however, that the cleaving treatment itself may constitute all or a portion of a swelling treatment when cleaving is carried out with a swelling reagent. For example, cleaving a peptide from the 2-CTC resin using dilute TFA in DCM would further constitute all or a portion of a swelling treatment. After cleaving, and after the swelling treatment is completed, the support is subjected to one or more shrinking washes that allow additional amounts of peptide to be recovered from such shrinking washes as well as enhancing the ability to recover additional peptide from one or more subsequent swelling washes. The subsequent swelling wash(es), constituting an additional swelling treatment, are carried out after the shrinking treatment is completed.

The peptide may be collected, further processed, etc. from the swelling washes and/or the shrinking washes using any desired techniques. For instance, the collected washes may be concentrated via distillation or the like. The peptide optionally may then be recovered from the concentrated mixture by any suitable technique although such isolation is not mandatory in all circumstances in which the peptide is to be further processed. An exemplary recovery technique involves precipitating the peptide, collecting the peptide via filtration, washing the collected peptide, and drying the peptide.

The methodologies of the present invention are very suitable with respect to a wide range of peptides synthesized via solid phase techniques. It is recognized, however, that the process may not be suitable for some peptide materials that are particularly temperature sensitive or otherwise degrade easily if not carefully handled, are too reactive, etc. Using conventional skills now or hereafter known in the art, simple empirical testing of peptide material can be undertaken to assess whether the candidate peptide material is compatible with the recovery process to the desired degree.

A preferred class of peptides of the present invention are those that incorporate from about 2 to about 100, preferably from about 4 to about 50, residues of one or more amino acids. Residues of one or more other monomeric, oligomeric, and/or polymeric constituents optionally may also be incorporated into a peptide. Non-peptide bonds also may be present. For instance, the peptides of the invention may be synthesized to incorporate one or more non-peptide bonds. These non-peptide bonds may be between amino acid residues, between an amino acid and a non-amino acid residue, or between two non-amino acid residues. These alternative non-peptide bonds may be formed by utilizing reactions well known to those in the art, and may include, but are not limited to imino, ester, hydrazide, semicarbazide, and azo bonds, to name but a few.

As used herein, the term “monomer” means a relatively low molecular weight material (i.e., generally having a molecular weight less than about 500 Daltons) having one or more polymerizable groups. “Oligomer” means a relatively intermediate sized molecule incorporating two or more monomers and generally having a molecular weight of from about 500 up to about 10,000 Daltons. “Polymer” means a relatively large material comprising a substructure formed two or more monomeric, oligomeric, and/or polymeric constituents and generally having a molecular weight greater than about 10,000 Daltons.

The amino acids from which the peptides are derived may be natural or non-natural. The twenty, common, naturally-occurring amino acids residues and their respective one-letter symbols are as follows: A (alanine); R (arginine); N (asparagine); D (aspartic acid); C (cysteine); Q (glutamine); E (glutamic acid); G (glycine); H (histidine); I (isoleucine); L (leucine); K (lysine); M (methionine); F (phenylalanine); P (proline); S (serine); T (threonine); W (tryptophan); Y (tyrosine); and V (valine). Naturally-occurring, rare amino acids are also contemplated and include, for example, selenocysteine, pyrrolysine.

Non-natural amino acids includes organic compounds having a similar structure and reactivity to that of a naturally-occurring amino acid include, for example, D-amino acids, beta amino acids, gamma amino acids; cyclic amino acid analogs, propargylglycine derivatives, 2-amino-4-cyanobutyric acid derivatives, Weinreb amides of α-amino acids, and amino alcohols. Incorporation of such amino acids into a peptide may serve to increase the stability, reactivity and/or solubility of the peptides of the invention.

The present invention contemplates that the recovered peptide material may act as intermediates in the synthesis of other peptides of interest through modification of the resultant peptide, through coupling of the peptide to other materials such as other peptides, or the like. For example, the present invention would be particularly useful to recover peptide fragment intermediates useful in the synthesis of enfuvirtide (also known as T-20), or alternatively DP-178. Such peptide fragments of the invention include, but are not limited to, those having amino acid sequences as depicted in Table 1 below:

TABLE 1

| | | |
|---|---|---|
| | | CORRE- |
| | | SPONDING |
| | | NUMBERED |
| | | AMINO |
| PEP- | | ACID |
| TIDE | | SEQUENCE |
| NO. | AMINO ACID SEQUENCE | OF T-20 |

| | | | |
|---|---|---|---|
| 1 | YTSLIHSL | (SEQ ID NO:2) | 1-8 |
| 2 | YTSLIHSLIEESQNQ | (SEQ ID NO:3) | 1-15 |
| 3 | YTSLIHSLIEESQNQQ | (SEQ ID NO:4) | 1-16 |
| 4 | YTSLIHSLIEESQNQQEK | (SEQ ID NO:5) | 1-18 |
| 5 | IEESQNQ | (SEQ ID NO:6) | 9-15 |
| 6 | IEESQNQQ | (SEQ ID NO:7) | 9-16 |
| 7 | QEKNEQELLELDKWASLWNW | (SEQ ID NO:8) | 16-35 |
| 8 | QEKNEQELLELDKWASLWNWF | (SEQ ID NO:9) | 16-36 |
| 9 | EKNEQEL | (SEQ ID NO:10) | 17-23 |
| 10 | EKNEQELLEL | (SEQ ID NO:11) | 17-26 |
| 11 | EKNEQELLELDKWASLWNWF | (SEQ ID NO:12) | 17-36 |
| 12 | NEQELLELDKWASLWNW | (SEQ ID NO:13) | 19-35 |
| 13 | NEQELLELDKWASLWNWF | (SEQ ID NO:14) | 19-36 |
| 14 | LELDKWASLWNW | (SEQ ID NO:15) | 24-35 |
| 15 | LELDKWASLWNWF | (SEQ ID NO:16) | 24-36 |
| 16 | DKWASLWNW | (SEQ ID NO:17) | 27-35 |
| 17 | DKWASLWNWF | (SEQ ID NO:18) | 27-36 |
| 18 | EKNEQELLELDKWASLWNW | (SEQ ID NO:19) | 17-35 |

Enfurvitide is a peptide that corresponds to amino acid residues 638 to 673 of the transmembrane protein gp41 from HIV-1.sub.LAI isolate and has the 36 amino acid sequence (reading from amino, NH₂ to carboxy, COOH, terminus):

(SEQ ID NO:1)

NH₂-YTSLIHSLIEESQNQQEKNEQELLELDKWASLWNWF-COOH

The chemical name of enfuvirtide is N-acetyl-Tyr-Thr-Ser-Leu-Ile-His-Ser-Leu-Ile-Glu-Glu-Ser-Gln-Asn-Gln-Gln-Glu-Lys-Asn-Glu-Gln-Glu-Leu-Leu-Glu-Leu-Asp-Lys-Trp-Ala-Ser-Leu-Trp-Asn-Trp-Phe-CONH₂. It will be understood that the principles of the present invention may also be applied in preferred embodiments to the recovery of peptides constituting all or a portion of T-20 and/or T-20-like peptide material. The term “T-20-like” as used herein includes any HIV or non-HIV peptide listed in U.S. Pat. Nos. 5,464,933; 5,656,480; 6,281,331; and 6,015,881; or PCT Publication No. WO 96/19495. The synthesis of peptides having T-20 activity and peptide intermediates used to prepare peptides having T-20 activity are described in U.S. Pat. Nos. 6,281,331; 6,015,881; 5,464,933; 5,656,480 and PCT Publication No. WO 96/19495.

In addition to peptides useful in the synthesis of enfuvirtide and enfuvirtide-like peptides, the principles of the present invention may be advantageously used to recover the following peptide material, fragment intermediates thereof, and/or analogs from a support after solid phase synthesis, especially when the material to be recovered is protected and FMOC synthesis has been used: Oxytocin (9 C SP); vasopressin: Felypressin, Pitressin (9 C), Lypressin (9 C), Desmopressin (9 C SP), Terlipressin (12 C); Atosiban (9 C); adrenocorticotropic hormone (ACTH; 24 C); Insulin (51 recombinant or semisynthesis), Glucagon (29 recombinant SP); Secretin (27); calcitonins: human calcitonin (32 C), salmon calcitonin (32 C SP), eel calcitonin (32 C SP), dicarba-eel (elcatonin) (31 C SP); luteinizing hormone-releasing hormone (LH-RH) and analogues: leuprolide (9 C), deslorelin (9 SP), triptorelin (10 SP), goserelin (10 SP), buserelin (9 SP); nafarelin (10 C), cetrorelix (10 SP), ganirelix (10 C), parathyroid hormone (PTH) (34 SP); human coriticotropin-releasing factor (41 SP), ovine coriticotropin-releasing factor (9 C SP); growth hormone releasing factor (9 C SP); somatostatin (9 C SP); lanreotide (9 C SP), octreotide (9 C SP); thyrotropin releasing hormone (TRH) (9 C SP); thymosin α-1(9 C SP); thymopentin (TP-5) (9 C SP); cyclosporin (9 C SP); integrilin (9 C SP); angiotensin-converting enzyme inhibitors: enalapril (9 C SP), lisinopril (9 C SP). In many embodiments, the peptide material to be recovered from a support is attached to the support at the carboxy end, while the N-terminus and side chain groups are protected, as appropriate, by protecting groups. The peptide desirably is cleaved under conditions such that undue loss of protection is avoided. The nature and use of protecting groups is well known in the art. Generally, a suitable protecting group is any sort of group that that can help prevent the atom or moiety to which it is attached, e.g., oxygen or nitrogen, from participating in undesired reactions during processing and synthesis. Protecting groups include side chain protecting groups and amino- or N-terminal protecting groups. Protecting groups can also prevent reaction or bonding of carboxylic acids, thiols and the like.

A side chain protecting group refers to a chemical moiety coupled to the side chain (i.e., R group in the general amino acid formula H₂N—C(R)(H)—COOH) of an amino acid that helps to prevent a portion of the side chain from reacting with chemicals used in steps of peptide synthesis, processing, etc. The choice of a side chain-protecting group can depend on various factors, for example, type of synthesis performed, processing to which the peptide will be subjected, and the desired intermediate product or final product. The nature of the side chain protecting group also depends on the nature of the amino acid itself. Generally, a side chain protecting group is chosen that is not removed during deprotection of the α-amino groups during the solid phase synthesis. Therefore the α-amino protecting group and the side chain protecting group are typically not the same.

In some cases, and depending on the type of reagents used in solid phase synthesis and other peptide processing, an amino acid may not require the presence of a side-chain protecting group. Such amino acids typically do not include a reactive oxygen, nitrogen, or other reactive moiety in the side chain.

Examples of side chain protecting groups include acetyl (Ac), benzoyl (Bz), tert-butyl, triphenylmethyl (trityl), tetrahydropyranyl, benzyl ether (Bzl) and 2,6-dichlorobenzyl (DCB), t-butoxycarbonyl (BOC), nitro, p-toluenesulfonyl (Tos), adamantyloxycarbonyl, xanthyl (Xan), benzyl, 2,6-dichlorobenzyl, methyl, ethyl and t-butyl ester, benzyloxycarbonyl (Z), 2-chlorobenzyloxycarbonyl (2-Cl-Z), Tos, t-amyloxycarbonyl (Aoc), and aromatic or aliphatic urethan-type protecting groups. photolabile groups such as nitro veritryl oxycarbonyl (NVOC); and fluoride labile groups such as trimethylsilyl oxycarbonyl (TEOC).

Preferred side chain protecting groups include t-Bu group for Tyr (Y), Thr (T), Ser(S) and Asp (D) amino acid residues; the trt group for His (H), Gln (O) and Asn (N) amino acid residues; and the Boc group for Lys (K) and Trp (W) amino acid residues.

For example, any one or more of the side-chains of the amino acid residues of peptide fragments listed in Table 1 may be protected with standard protecting groups such as t-butyl (t-Bu), trityl (trt) and t-butyloxycarbonyl (Boc). The t-Bu group is the preferred side-chain protecting group for amino acid residues Tyr (Y), Thr (T), Ser(S) and Asp (D); the trt group is the preferred side-chain protecting group for amino acid residues His (H), Gln (O) and Asn (N); and the Boc group is the preferred side-chain protecting group for amino acid residues Lys (K) and Trp (W).

During the synthesis of fragments of Table 1 that include histidine, the side-chain of the histidine residue desirably is protected, preferably with a trityl (trt) protecting group. If it is not protected, the acid used to cleave the peptide fragment from the resin and/or to cleave FMOC or other N-terminal protecting groups during synthesis could detrimentally react with an unprotected histidine residue, causing degradation of the peptide fragment. Quite possibly, no further attachment of another amino acid could occur if histidine is not protected. Extended cleavage time also may remove a protecting group such as trt from histidine and can cause a batch to satisfy typical quality specifications.

Preferably, the glutamine residues of the peptide fragments of the invention are protected with trityl (trt) groups. However, it is preferred not to protect the glutamine residue at the carboxy-terminal end of fragments 1-16 and 9-16. The absence of a protective group on the glutamine residue at the carboxy-terminal end of the 1-16 fragment facilitates reaction of the 1-16 fragment with the 17-36 fragment, allowing coupling of the fragments with only about 2% racemization. In addition, if lower solubility of any of the peptide fragments of the invention in organic solvents is desired, the trityl protecting groups may be eliminated from any one or more of the other glutamine residues of the fragments.

Preferably, all the asparagine residues of each peptide fragment of the invention are protected. In addition, it is preferred that the tryptophan residue is protected with a Boc group.

An amino-terminal protecting group includes a chemical moiety coupled to the alpha amino group of an amino acid. Typically, the amino-terminal protecting group is removed in a deprotection reaction prior to the addition of the next amino acid to be added to the growing peptide chain, but can be maintained when the peptide is cleaved from the support. The choice of an amino terminal protecting group can depend on various factors, for example, type of synthesis performed and the desired intermediate product or final product.

Examples of amino-terminal protecting groups include (1) acyl-type protecting groups, such as formyl, acrylyl (Acr), benzoyl (Bz) and acetyl (Ac); (2) aromatic urethan-type protecting groups, such as benzyloxycarbonyl (Z) and substituted Z, such as p-chlorobenzyloxycarbonyl, p-nitrobenzyloxycarbonyl, p-bromobenzyloxycarbonyl, p-methoxybenzyloxycarbonyl; (3) aliphatic urethan protecting groups, such as t-butyloxycarbonyl (BOC), diisopropylmethoxycarbonyl, isopropyloxycarbonyl, ethoxycarbonyl, allyloxycarbonyl; (4) cycloalkyl urethan-type protecting groups, such as 9-fluorenyl-methyloxycarbonyl (Fmoc), cyclopentyloxycarbonyl, adamantyloxycarbonyl, and cyclohexyloxycarbonyl; and (5) thiourethan-type protecting groups, such as phenylthiocarbonyl. Preferred protecting groups include 9-fluorenyl-methyloxycarbonyl (Fmoc), 2-(4-biphenylyl)-propyl(2)oxycarbonyl (Bpoc), 2-phenylpropyl(2)-oxycarbonyl (Poc) and t-butyloxycarbonyl (Boc).

Any type of support suitable in the practice of solid phase peptide synthesis can be used. In preferred embodiments, the support comprises a resin that can be made from one or more polymers, copolymers or combinations of polymers such as polyamide, polysulfamide, substituted polyethylenes, polyethyleneglycol, phenolic resins, polysaccharides, or polystyrene. The polymer support can also be any solid that is sufficiently insoluble and inert to solvents used in peptide synthesis so long as the solid support is able to shrink and swell when contacted with a suitable reagent. The solid support typically includes a linking moiety to which the growing peptide is coupled during synthesis and which can be cleaved under desired conditions to release the peptide from the support. Suitable solid supports can have linkers that are photo-cleavable, TFA-cleavable, HF-cleavable, fluoride ion-cleavable, reductively-cleavable; Pd(O)-cleavable; nucleophilically-cleavable; or radically-cleavable. Preferred linking moieties are cleavable under conditions such that the cleaved peptide is still substantially globally protected.

Preferred solid supports include acid sensitive solid supports, for example, hydroxymethyl-polystyrene-divinylbenzene polymer (“Wang” resins; Wang, S. S. 1973, J. Am. Chem. Soc., 95: 1328-33), 2-chlorotrityl chloride resin (see, e.g., Barlos et al., 1989, Tetrahedron Letters 30(30): 3943-3946) and 4-hydroxymethyl methoxyphenoxybutyric acid resin (see, e.g., Richter et al., 1994, Tetrahedron Letters 35(27): 4705-4706), functionalized, crosslinked poly N-acrylylpyrrolidine, and chloromethylpolystyrene divinylbenzene polymer. These types of solid supports are commercially available from, for example, Calbiochem-Novabiochem Corp., San Diego, Calif.

In some embodiments, a preferred support includes acid sensitive supports such as 2-chlorotrityl chloride resin (2-CTC) or CT2 preloaded with the first amino acid. Peptide material typically is attached to the resin beads both at the bead surfaces and within the bead interiors. FMOC and side chain protected peptide is readily cleaved in a protected state from this resin using mildly acidic reagents such as dilute TFA in DCM or acetic acid. The cleaving reaction is clean, allowing cleaved peptide to be recovered in a straightforward manner from the cleaving reagent as well as from the follow up shrinking and swelling washes.

In one embodiment, a swelling reagent generally refers to a liquid reagent that causes the volume of dried resin beads to swell by at least 40%, preferably at least 50%, and more preferably at least 100% in volume when one gram of beads is immersed in about 10 ml of the reagent at a temperature in the range of −15° C. to 25° C. for a time period in the range of from about 2 minutes to about 50 minutes. In many cases, a swelling reagent may cause the bead volume to at least double or even triple in volume.

Alternatively stated, one embodiment of a swelling solvent is characterized by a swelling factor of at least 1.4, preferably at least about 1.8, where the swelling factor is given by the ratio of the final resin volume after immersion to the initial dried resin volume prior to immersion.

Selection of one or more appropriate swelling reagents will depend to a large degree upon the nature of the support. For instance, if the resin is generally polar in nature, then a suitable swelling reagent will tend to be polar as well. If the resin is generally nonpolar, then a suitable swelling reagent will tend to be nonpolar. For example, the 2-CTC resin is a generally nonpolar, styrene based resin that is swelled by nonpolar solvents such as, for example, dicholoroethane (DCE), dichloromethane (DCM), chloroform, benzene, toluene, acetone, tetrohydrofuran, N-methylpyrrolidinone (MNP), or the like. Support materials swell in these types of solvents to multiple times their dry volume. Preferred swelling reagents include chlorinated liquids such as one or more of DCE, DCM, dichloromethane, chloroform, or the like. DCM is presently preferred. Although DCE provides greater loading efficiency according to literature sources, DCM may be substituted with little or no reduction in the loading.

In one embodiment, a shrinking reagent generally refers to a liquid reagent that causes the volume of dried resin beads to increase in volume by at most about 40%, preferably at most about 30%, more preferably at most about 15% when about 1 gram of the resin is immersed in about 10 ml of the reagent at a temperature in the range of −15° C. to 25° C. for a time period in the range of from about 2 minutes to about 50 minutes. Alternatively stated, one embodiment of a shrinking solvent has a swelling factor of at most about 1.4, preferably at most about 1.3, more preferably at most about 1.15. Note that immersion of swelled resin in one or more “shrinking” reagents causes a shrinking effect to occur.

Selection of one or more appropriate shrinking reagents will depend to a large degree upon the nature of the resin support. For instance, if the resin is generally polar in nature, then a suitable shrinking reagent will tend to be relatively nonpolar. If the resin is generally nonpolar, then a suitable shrinking reagent will tend to be relatively polar. For example, the 2-CTC resin is shrunk by reagents such as methanol, ethanol, ether, isopropyl alcohol, hexane, or the like. Preferred shrinking reagents include alcohols such as ethanol, isopropyl alcohol, combinations of these, or the like.

The swelling factors of representative swelling and shrinking reagents are listed below with respect to H-Leu-2-CTC resin (a polystyrene-based resin):

| Reagent | Swell Factor |
|---|---|
| DCM | 3.5 |
| Chloroform | 3.5 |
| THF | 3.3 |
| NMP | 3.2 |
| Toluene | 3.0 |
| DMF | 2.8 |
| Acetone | 2.2 |
| Ethanol 3C | 1.2 |
| Methanol | 1.1 |
| Isopropanol | 1.1 |
| Hexane | 1.1 |

As noted above, some embodiments of the invention may involve selecting a swelling reagent having a swell factor above a threshold level and a shrinking reagent having a swell factor below a certain threshold level. In alternative embodiments, the swelling and shrinking reagents may be selected not on such absolute criteria but rather based on the relative differences between the swelling factors of these reagents. In other words, so long as the relative difference between the swell factors of the reagents is large enough, a shrink/swell effect of the present invention may still be achieved.

Thus, in such alternative embodiments, first and second immersion reagents are selected such that the ratio of the swell factor of the first reagent (which functions as the swelling reagent) to the second reagent (which functions as the shrinking reagent) is at least about 1.3, more preferably at least about 1.8, more preferably at least about 2. In this way, the first reagent functions as a swelling reagent relative to the second reagent, which functions as the shrinking agent relative to the first reagent.

In a particularly preferred embodiment, the support comprises the 2-CTC resin, the swelling wash comprises a chlorinated, nonpolar solvent such as DCM, and the shrinking reagent comprises a mixture of ethanol and isopropyl alcohol, especially a solution containing about 5 parts by weight IPA per 95 parts by weight ethanol. This combination has been effective when recovering peptides such as the peptides according to Seq. ID. Nos. 11 and 17 listed above after solid phase synthesis. The shrinking wash solution may additionally contain a small amount of water, but not so much such that phase separation results when combining the shrinking wash solution with the nonpolar swelling solvent. For instance, in the absence of water, DCM is readily soluble in an ethanol/PA solution. Peptide fragments such as those according to Seq. ID. Nos. 11 and 17 listed above used to make enfuvirtide are readily soluble in DCM, but only sparingly solvent in ethanol/IPA. For example, in one case, a resin sample after shrinking was split into two equal parts. One sample was washed with dichloromethane, the other with ethanol. The amount of material extracted into dichloromethane was about 20 times that extracted into ethanol. Because of the high solubility of the peptide fragments in DCM, however, the fragments are highly soluble in miscible mixtures of DCM/ethanol/IPA, even when the amount of DCM is relatively low, e.g., as low as 2 to 5% by weight of the total DCM/ethanol/IPA. Consequently, precipitation of peptide material in such compositions may be achieved by addition of a third, polar liquid such as water or the like.

A representative process embodiment to recover peptide material from a support resin will now be described using a swell/shrink/swell (A-B-A) strategy. An additional shrink treatment (B) optionally may be practiced after the last swelling treatment (A) to make it easier to process the resin support for disposal or recycling. The representative apparatus used for peptide recovery in the illustrative process includes a vessel in which the support, desirably in the form of a bed of resin beads, is held during the wash treatments to be described. The vessel includes an outlet through which liquids are withdrawn. Typically, a filter is used to help retain beads in the vessel when the liquid is withdrawn. Additionally, the vessel includes agitation componentry to agitate the vessel contents during wash treatments. The agitation componentry typically is above the beads to avoid unduly damaging the beads. The vessel may be pressurized to help push liquid out of the vessel through the filter. As the washes are forced out of the vessel by the pressure, the filter retains the resin beads. An inert gas such as nitrogen, carbon dioxide, argon, or the like, may be used for such pressurization or vacuum applied below the filter.

At the outset, the beads supporting the peptide material to be recovered are positioned in the vessel. Optionally, if there is a chance that the beads may incorporate residual NMP or the like, the beads may be washed one or more times with a nonpolar, chlorinated solvent such as DCM, DCE, chloroform, or the like. Such washing helps to remove the NMP, or similar constituent, that can react with acidic cleaving reagents.

If the peptide material has not yet been cleaved from the resin, a cleaving treatment is carried out in a manner such that the cleaved peptide still bears sufficient side chain and terminus protecting groups. Leaving the protective groups in place helps to prevent undesirable coupling or other undesirable reactions of peptide fragments during or after cleaving. In the case when FMOC or similar chemistry is used to synthesize the peptide, protected cleaving may be accomplished in any desired fashion such as by using a relatively weak acid reagent such as acetic acid or dilute TFA in a swelling solvent such as DCM. The use of 0.5 to 10 weight percent, preferably 1 to 3 weight percent TFA in DCM is preferred. See, e.g., U.S. Pat. No. 6,281,335.

To accomplish cleaving, approximately 5 to 20, preferably about 10 volumes of the acidic cleaving reagent is added to the vessel. The resin beads are immersed in the reagent as a consequence. The cleaving reaction occurs as the liquid contents are agitated at a suitable temperature for a suitable time period. Agitation helps prevent the beads from clumping. Suitable time and temperature conditions will depend upon factors such as the acid reagent being used, the nature of the peptide, the nature of the resin, and the like. As general guidelines, stirring at from about −15° C. to about 5° C., preferably from about −10° C. to about 0° C. for about 5 minutes to two hours, preferably about 25 minutes to about 45 minutes would be suitable. Cleaving time may be in the range of from about 10 minutes to about 2 hours. For large-scale production, a preferred time is in the range of from about 15 to 50 minutes. Cleaving is desirably carried out in such chilled temperature range to accommodate a reaction exotherm that might typically occur during the reaction.

At the end of the cleaving treatment, the reaction is quenched. This may be achieved, for example, by adding a suitable base, such as pyridine or the like, to the vessel, and continuing to agitate and stir for an additional period such as for an additional 5 minutes to 2 hours, preferably about 20 minutes to about 40 minutes. Adding the base and continued agitation causes the temperature of the vessel contents to increase. At the end of agitation, the vessel contents may be at a temperature in the range of from about 0° C. to about 15° C., preferably about 5° C. to about 10° C.

Because a swelling solvent such as DCM may be used as a constituent in the cleaving reagent, the cleaving treatment also may constitute a first swelling treatment in which a significant amount of cleaved peptide will be extracted into the liquid. When swelled with TFA in DCM, the bead volume will tend to be largest at the onset of the cleaving treatment. The beads will still be swelled, but their volume decreases, as peptide is extracted into the liquid.

After quenching, the vessel contents are emptied and collected to recover the peptide extracted into the wash. Pressure may be used to force the liquid mixture containing peptide material carried by the liquid through the filter and out of the vessel. The beads remaining in the vessel will still contain residual DCM and will still be swelled to some extent. A significant amount of residual peptide also tends to be retained in the beads, and the subsequent shrinking and swelling treatments help to recover significant portions of the residual peptide.

As an option, it may be desirable to wash the collected cleaving reagent with water prior to concentration via distillation or the like, usually after some concentration has been accomplished except. Water washing after cleaving is believed to be useful to enhance peptide quality and, therefore, to some extent yield. For instance, increased contact time with the TFA and other ingredients in the cleaving reagent might impair peptide quality such as by detritylation at His 6 and/or esterification of the peptide. Water washing is believed to be helpful in removing residual TFA and its byproducts. After the water wash/extraction treatment, the liquid mixture may be transferred to a distillation apparatus, where the mixture is concentrated further by removing, for example, the DCM or the like. Some fragments, such as a peptide according to Seq. ID. No. 4 of Table 1, may not benefit from water washing due to sensitivity or other characteristic of the peptide material.

After the cleaving mixture is emptied from the vessel and collected for peptide recovery, the vessel contents may be subjected to one or more additional swelling washes into which additional peptide may be extracted and then recovered. These additional swelling washes also help to wash the vessel and remove residual cleaving reagents and by-products. Such ingredients are desirably removed prior to proceeding with a shrinking treatment so that the shrinking liquid will not react with them. For instance, it is desirable to remove TFA from the vessel before adding a shrinking liquid containing ethanol inasmuch as ethanol can react with TFA. A typical swelling wash treatment may occur with agitation for a time period of from about 2 minutes to 2 hours, preferably from about 10 minutes to about 50 minutes. After the wash is done, the wash is removed from the vessel and then may be added to the distillation vessel with the other swelling washes. Optionally, prior to being added to the distillation pot, these additional swelling washes, if any, may be subjected to a water extraction treatment to remove impurities.

The vessel contents may now be subjected to a shrinking treatment comprising subjecting the vessel contents to one or more shrinking washes. The peptide material may be insoluble, soluble, or only sparingly soluble in such shrinking liquid. As used herein, insoluble preferably means that more than about 70%, preferably more than 95% by weight of 10 parts by weight or more of peptide exists as a precipitate in at least about 50, preferably at least about 100 parts by weight, more preferably at least about 500 parts by weight of the liquid at 25° C.; soluble means that more than about 70%, preferably more than about 95% by weight of 10 parts by weight or more of peptide is dissolved in at least about 50, preferably at least about 100, more preferably at least about 500 parts by weight of the liquid at 25° C.; and sparingly soluble means that from about 30% to about 70%, preferably 5% to about 95% by weight of about 10 parts by weight or more of peptide is dissolved in at least about 50, preferably at least about 100, more preferably at least about 500 parts by weight of the liquid at 25° C. The volume of shrinking liquid used is not critical, although it is desirable to ensure that enough is used to allow the beads to be fully submerged and to allow the liquid phase to be stirred during the treatment. Typically, about 4 to about 6 volumes of the shrinking liquid per volume of the beads would be suitable. This treatment may occur over a wide range of time and temperature conditions, but conveniently would be carried out at room temperature for a period in the range of from about 5 to about 60 with stirring.

This wash causes the resin beads to shrink in size relative to their swelled condition. The shrinkage preferably is substantial enough to be visually observed as a reduction in the bed depth of the beads. While not wishing to be bound by a particular theory, it appears that such liquid-induced shrinkage causes additional, recoverable peptide material to be extruded from the resin beads or at least to be extruded closer to the resin surfaces where extraction with a subsequent swelling wash is easier. Consequently, the use of the shrinking reagent helps to increase recovery yield by helping to make more resin available for recovery.

During the shrinking wash, not just peptide but also residual swelling solvent, such as DCM if that was used, tends to be extruded from the beads as well. Thus, it is desirable that the shrinking and swelling compositions be soluble in each other. This is one reason why DCM is preferred as a swelling reagent while ethanol/IPA is a preferred shrinking mixture, inasmuch as the liquid compositions are readily soluble in one another. However, if the overall water content in the ethanol/IPA mixture is too high, more than one liquid phase may result when the DCM intermixes with the ethanol/IPA/water. Phase separation is desirably avoided by ensuring that the overall water content is low enough, e.g., less than 3 weight percent, more preferably less than about 1 weight percent. Because the shrinking solvent may be substantially a non-solvent, or poor solvent, for the peptide material, the swelling solvent extruded at this point helps to carry peptide material into the bulk liquid.

At the end of the shrinking wash, the liquid contents of the vessel are removed through the filter and collected in a separate vessel. Peptide may be recovered from this wash. However, these contents are not yet sent to the distillation apparatus. If the liquid were sent directly to the distillation apparatus at this stage, the alcohol in the liquid could react with the peptide material, yielding undesirable ester embodiments of the peptides. At a later stage, usually after water washing of the DCM stream, however, such materials will be further handled and sent to the distillation, and that further handling of the collected mixture will be described further below.

Optionally, one or more additional shrinking washes may be performed as desired.

After performing the one or more shrinking washes, the beads are subjected to at least one additional swelling wash with a swelling liquid such as DCM. The amount of swelling liquid, the temperature, the residence time, the agitation, and other treatment conditions may be as described above with respect to the previous DCM washes performed after peptide cleaving. Additional peptide material is typically extracted at this stage. When the wash is complete, the wash liquid is transferred to the distillation apparatus.

At this point, analysis of the beads may be carried out to determine how much peptide still remains in the beads. If the resin still contains above a threshold amount, of peptide, e.g., more than 10-15 g peptide per kg resin, an additional shrinking/swelling cycle can be carried out to recover more peptide from the beads after which the beads may again be analyzed to see how much peptide remains in the beads. When all desired treatments are done, the beads may be further processed, recycled, discarded, or the like, while as much peptide as practically possible is recovered from the various washes.

Either a swelling treatment or a shrinking treatment may be the last treatment to be carried out on the beads. However, when the shrinking liquid is an ethanol/IPA mixture, it is desirable for practical reasons to finish with a shrinking treatment. Quite simply, ethanol and IPA are relatively easy to remove from the beads, and this makes bead disposal easier and less expensive.

With the last treatment being completed, typically the swelling washes have been transferred to the distillation apparatus while the shrinking washes have been transferred to a holding vessel or the like. A wide variety of distillation conditions may be used. As one example, distillation may occur at a temperature in the range of from about 5° C. to about 25° C., preferably about 10° C. to about 20° C. and a vacuum of about 50 to 500 mmHg, preferably about 100 to 300 mmHg.

Distillation is continued on the swelling washes until the DCM volume is stripped down to a desired volume at which it is safe to add the shrinking washes without undue risk that peptide esters could form. When the desired volume is reached, the shrinking washes may be added to the distillation apparatus. Distillation continues but then is stopped when the concentration of the swelling solvent in the shrinking solvent reaches a desired level. Stopping the distillation at an appropriate point can be important in some embodiments, inasmuch as the relative amounts of shrinking and swelling reagents remaining in the concentrate can impact further processability. For instance, if the peptide is to be recovered from the concentrate via precipitation, filtering (with washes), and drying, the concentration of DCM relative to ethanol/IPA can impact the filtering characteristics of the precipitate. If the amount of DCM is too high or too low, the precipitate may be too fine, too coarse, or too tacky. This target concentration at which distillation is optimally stopped will vary depending upon factors such as the nature of the peptide, the nature of the shrinking liquid, the nature of the swelling liquid, and the like. For example, the desired amount of swelling reagent remaining in the concentrate when distillation is stopped may be determined experimentally. Generally, data is gathered that indicates the filtering characteristics of the precipitated peptide as a function of the DCM content in the concentrate. The data typically will show a swelling reagent content that works well.

For instance, when recovering a peptide according to Seq. ID. No. 11 from a concentrate of combined DCM washes and ethanol/IPA washes, distillation desirably is stopped when the mixture being distilled includes from 4 to 8 percent by weight DCM. When recovering a peptide according to Seq. ID. No. 17 from a concentrate of combined with DCM washes and ethanol/IPA washes, distillation desirably is stopped when the mixture being distilled includes from 4 to 8 percent by weight DCM.

At the end of distillation, the peptide tends to still largely be in solution (but it could be hazy or some small amount of precipitate may be present), even though the mixture may include a relatively large amount of shrinking liquid such as ethanol/IPA in which the peptide is only sparingly soluble or perhaps insoluble. This solubility occurs because the peptide may be very soluble in a swelling liquid such as DCM. Consequently, the peptide is then generally recovered from the concentrate via a suitable recovery technique such as precipitation, filtering, and drying. Precipitation of the peptide may be induced by adding a suitable precipitating liquid to the concentrate with moderate agitation. The precipitating liquid may be added in one or more aliquots. After each aliquot is added, the DCM volume may be further reduced by vacuum distillation if desired. Precipitation may be accomplished in any suitable fashion such as by slowly adding an aqueous composition to the mixture. The precipitated peptide may then be collected by filtration. It is generally desirable to wash the filtered peptide one or more times. The peptide can then be dried and qualified for further handling, processing, etc.

To sum up this preferred process embodiment of the invention involves an alternating sequence comprising swell-shrink-swell-shrink treatments. The beads have been washed one or more times when swelled and then washed one or more times when shrunk. The process then continues by alternating back to a swelling phase comprising one or more swelling washes to extract additional peptide. As noted above, finishing the alternating sequence with a shrink treatment is a convenience in terms of processing the beads for disposal, recycling, or the like. As an option, additional swell-shrink cycles may be practiced depending upon the amount of peptide remaining in the resin after the last treatment.

Additional procedures involved in the solid phase, solution phase, and/or hybrid synthesis of peptides are discussed in the following U.S. provisional applications: (1) U.S. provisional application No. 60/533,653, filed Dec. 31, 2003, titled “Process and Systems for Recovery of Peptides” bearing attorney docket no. RCC0009/P1, in the names of inventors including Hiralal Khatri; (2) U.S. provisional application No. 60/533,691, filed Dec. 31, 2003, titled “Peptide Synthesis Using Filter Decanting” bearing attorney docket no. RCC0010/P1, in the names of inventors including Mark A. Schwindt; (3) U.S. provisional application No. 60/533,654, filed Dec. 31, 2003, titled “Process and Systems for Peptide Synthesis” bearing attorney docket no. RCC0011/P1, in the names of inventors including Mark A. Schwindt; and (4) U.S. provisional application No. 60/533,710, filed Dec. 31, 2003, titled “Peptide Synthesis and Deprotection Using a Cosolvent” bearing attorney docket no. RCC0012/P1, in the names of inventors including Mark A. Schwindt.

The present invention will now be further described with reference to the following illustrative examples.

### EXAMPLE 1

### Cleavage and Isolation of Fmoc(17-26)OH from its Polymeric Support (Using Swell/Shrink Strategy)

Fragment 2 (Fmoc(17-26)OH refers to a globally protected peptide according to Seq ID No. 11 in which the N-terminus bears an FMOC protecting group, side group protecting groups, and no protecting group at the C-terminus after being cleaved from the resin support (i.e., fully protected FmocLELLEQENKE). Fragment 2 was assembled on a 2-chlorotrityl chloride functionalized bead composed of polystyrene (PS), crosslinked with 1% divinyl benzene and preloaded with the first amino acid, Fmoc-LeuOH (17 kg loaded resin, leucine loading 0.76 mmol/g).

During the course of synthesis, the removal of the Fmoc protecting group, coupling reactions and other aspects of Fmoc/t-Butyl solid phase synthesis are consistent with general methodologies known to one skilled in the art and embodied in such publications as “Solid Phase Peptide Synthesis—A Practical Approach” by E. Atherton and R. C. Sheppard, IRL Press at Oxford University, 1989, ISBN 1 85221 134 2 and ISBN 1 85221 133 4 and more recently in monographs such as “Fmoc Solid Phase Peptide Synthesis—A Practical Approach” by W. C. Chan and P. D. White, Oxford University Press, 2000, ISBN 0 19 963 725 3 and ISBN 0 19 963 725 4. See also U.S. Pat. No. 6,281,331.

The solid phase synthesis/filtration vessel, containing the resin-bound peptide, was cooled to 0 to −10° C. and dichloromethane 216 kg, 9-10 volumes, was charged to a second vessel. Trifluoroacetic acid (TFA, 5.0 kg, 0.2 volumes relative to resin weight) was added to the vessel containing the dichloromethane to give a concentration of ˜2% TFA in dichloromethane. This solution was then cooled to 0 to −10° C. with moderate agitation (final temperature −4.5° C.).

The acid solution was then transferred from the second vessel to the synthesis vessel containing the resin-bound peptide and the transfer line flushed with dichloromethane (8.5 kg, 0.38 volumes relative to resin weight). The resin was rapidly agitated to prevent clumping in the synthesis/filtration vessel and to facilitate cleavage of the peptide from the resin. The temperature was held at about −10° C. to +2° C. for at least 30 min (Actual=30 min exposure time and 1.0° C. final temperature). The cleavage reaction was terminated by the addition of pyridine (4.0 kg, 0.24 volumes, 1.2 equiv relative to TFA) directly into the synthesis/filtration vessel. The synthesis/filtration vessel jacket was set at 10° C. and the vessel purged with nitrogen. The vessel was then agitated for at least 5 min at a temperature of 5 to 15° C. (Actual values 7 min stir, final temperature 8° C.). The cleavage solution was transferred to a third vessel for distillation. Transfer to the third vessel could be facilitated either by applying vacuum to the third vessel and/or by applying pressure to the synthesis/filtration vessel.

A dichloromethane wash (113 kg, 5 to 6 volumes) was sent to the synthesis/filtration vessel and the mixture agitated for 30 min at room temperature then sent to the third distillation vessel. Concentration of the combined cleavage solution and DCM wash by vacuum distillation was initiated at this point while maintaining the vessel temperature at 10 to 20° C. and the vessel jacket temperature below 40° C. Vacuum was varied to maintain these parameters. Ethanol (133 kg, 183 L) was stored in the second vessel at room temperature. The first ethanol wash ˜100 L was sent to the synthesis/filtration vessel where the mixture was agitated for at least 15 min and then sent to a dedicated holding tank.

In the meantime, concentration of the cleavage solution and dichloromethane was continued using vacuum distillation keeping the vessel temperature 10 to 20° C. and the vessel jacket temperature below 40° C. Vacuum was varied to maintain these parameters. Vacuum distillation was continued until a specified volume is reached (typically ˜25% of the original cleavage solution volume).

One further ethanol wash ˜83 L was sent to the synthesis/filtration vessel where the mixture was agitated for at least 15 min and then the wash was sent to the dedicated holding tank. The combined ethanol washes were then sent to the distillation vessel and distillation continued under vacuum until a dichloromethane in ethanol concentration of 3 to 10% is attained (Actual 5.7% DCM in ethanol).

Residual, non-cleaved fragment left on resin at cleavage completion was 5.5 g/Kg (94 g) or 0.4% of the total isolated peptide. At the end of the wash cycle the cleaved peptide still washing out the resin was not quantified.

Water (85 L, 5 volumes) was slowly charged to the distillation vessel with moderate to rapid agitation over 30 min while maintaining a temperature of 9 to 15° C. during the addition. Using moderate to slow agitation, the precipitating peptide slurry was cooled to −5 to 5° C. over at least 1 hour (Actual=2.3 h).

The precipitated peptide was then isolated in an agitated filter vessel, pre-cooled to −5 to 5° C., agitated with 2 volumes of water and filtered, then agitated with 5 to 10 volumes of ethanol/water mixture (80:20) which had been pre-cooled to −5 to 5° C. and filtered. The product was blown dry with an inert gas such as nitrogen and either dried in the filter or removed from the filter and dried in an appropriate drying apparatus. Drying was continued at <100 mmHg and a jacket temperature of <45° C. until the water content of the peptide was <1%.

A yield of 21.8 kg (74.5% Theory) was obtained, theoretical yield was 29.3 kg, and Purity 95.2% by area normalized HPLC.

### EXAMPLE 2

### Cleavage and Isolation of Fmoc(17-26)OH from Its Polymeric Support (Swell/Shrink/Swell/Shrink Strategy)

Fragment 2 (Fmoc(17-26)OH, Fully protected FmocLELLEQENKE) was assembled on a 2-chlorotrityl chloride functionalized bead composed on polystyrene (PS), crosslinked with 1% divinyl benzene and pre-loaded with the first amino acid, Fmoc-LeuOH (21 kg loaded resin, leucine loading 0.70 mmol/g).

The removal of the Fmoc protecting group, coupling reactions and other aspects of Fmoc/t-Butyl solid phase synthesis are consistent with general methodologies known to one skilled in the art and embodied in such publications as “Solid Phase Peptide Synthesis—A Practical Approach” by E. Atherton and R. C. Sheppard, IRL Press at Oxford University, 1989, ISBN 1 85221 134 2 and ISBN 1 85221 133 4 and more recently in monographs such as “Fmoc Solid Phase Peptide Synthesis—A Practical Approach” by W. C. Chan and P. D. White, Oxford University Press, 2000, ISBN 0 19 963 725 3 and ISBN 0 19 963 725 4. See also U.S. Pat. No. 6,281,331.

The solid phase synthesis/filtration vessel containing the resin-bound peptide was cooled to 0 to −10° C. and dichloromethane 267 kg, 9-10 volumes, was charged to a second vessel. Trifluoroacetic acid (TFA, 6.2 kg, 0.2 volumes relative to resin weight) was added to the vessel containing the dichloromethane to give a concentration of ˜2% TFA in dichloromethane. This solution was then cooled to 0 to −10° C. with moderate agitation (final temperature −9.2° C.).

The acid solution was then transferred from the second vessel to the vessel containing the resin-bound peptide and the transfer line flushed with dichloromethane (10.5 kg, 0.38 volumes relative to resin weight). The resin was rapidly agitated in the synthesis/filtration vessel to prevent clumping and to facilitate cleavage of the peptide from the resin. The temperature was held at ˜−10 to +2° C. for at least 30 min (Actual=30 min exposure time and 0.1° C. final temperature). The cleavage reaction was terminated by the addition of pyridine (4.9 kg, 0.24 volumes, 1.2 equiv relative to TFA) directly into the synthesis/filtration vessel. The synthesis/filtration vessel jacket was set at 10° C. and the vessel purged with an inert gas such as nitrogen. The vessel was then agitated for at least 5 min at a temperature of 5 to 15° C. (Actual values 10 min stir, final temperature 7° C.). The cleavage solution was transferred to a third vessel for distillation.

A dichloromethane wash (139 kg, 5 to 6 volumes) was sent to the synthesis/filtration vessel and the mixture agitated for 30 min at room temperature then sent to the third distillation vessel. Concentration of the combined cleavage solution and dichloromethane wash by vacuum distillation was initiated at this point while maintaining the vessel temperature at 10 to 20° C. and the jacket temperature below 40° C. Vacuum was varied to maintain these parameters.

Ethanol (273 kg, 375 L) was stored in the second vessel at room temperature. The first ethanol wash ˜120 L was sent to the synthesis/filtration vessel where the mixture was agitated for at least 15 min and then sent to holding tank.

A second dichloromethane wash (139 kg, 5 to 6 volumes) was sent to the synthesis/filtration vessel and the mixture agitated for 30 min at room temperature then sent to the third distillation vessel. Concentration of the combined cleavage solution and dichloromethane washes by vacuum distillation was continued at this point while maintaining the vessel temperature at 10 to 20° C. and the jacket temperature below 40° C. Vacuum was varied to maintain these parameters.

Concentration of the cleavage solution and dichloromethane was continued using vacuum distillation keeping the vessel temperature 10 to 20° C. and the jacket temperature below 40° C. Vacuum was varied to maintain these parameters.

Vacuum distillation was continued until a specified volume is reached (typically ˜25% of the original cleavage solution volume).

Two further ethanol washes ˜110 to 130 L were sent to the synthesis/filtration vessel where the mixture was agitated for at least 15 min and then the washes were sequentially sent to holding tank. The combined ethanol washes were then sent to the distillation vessel and distillation continued under vacuum until a dichloromethane in ethanol concentration of 3 to 10% is attained (Actual 3.5% DCM in ethanol).

Residual non-cleaved fragment left on resin at cleavage completion was negligible. No fragment 2 was detected. At the end of the wash cycle the cleaved peptide still washing out the resin amounted to 5.21 g/kg (109.41 g) or 0.4% of the weight of isolated material.

Water (105 L, 5 to 6 volumes) was slowly charged to the distillation vessel with moderate to rapid agitation over 30 min maintaining a temperature of 9 to 15° C. during the addition. Using moderate to slow agitation the precipitating peptide slurry was cooled to −5 to 5° C. over at least 1 hour (Actual=2.5 h).

The precipitated peptide was then isolated in an agitated filter vessel, pre-cooled to −5 to 5° C., agitated with 2 volumes of water and filtered. It was then further agitated with 5 to 10 volumes of ethanol/water mixture (80:20) which had been pre-cooled to −5 to 5° C. and filtered. The product was blown dry with an inert gas such as nitrogen and either dried in the filter or removed from the filter and dried in an appropriate drying apparatus. Drying was continued at <100 mmHg and a jacket temperature of <45° C. until the water content of the peptide was <1%.

A yield of 24.3 kg (72.5% Theory) was obtained, theoretical yield was 33.45 kg, and purity was 95.4% by area normalized HPLC.

This procedure was repeated for 4 additional batches. Over the five batches, the following data was obtained: average yield was 79.5%; average purity was 94.1%.

### EXAMPLE 3

### Cleavage and Isolation of Fmoc(17-26)OH from Its Polymeric Support (Swell/Shrink/Swell/Shrink)

Fragment 2 (Fmoc(17-26)OH, Fully protected FmocLELLEQENKE) was assembled on a 2-chlorotrityl chloride functionalized bead composed on polystyrene (PS), crosslinked with 1% divinyl benzene and preloaded with the first amino acid, Fmoc-LeuOH (24 kg loaded resin, leucine loading 0.85 mmol/g).

The removal of the Fmoc protecting group, coupling reactions and other aspects of Fmoc/t-Butyl solid phase synthesis are consistent with general methodologies known to one skilled in the art and embodied in such publications as “Solid Phase Peptide Synthesis—A Practical Approach” by E. Atherton and R. C. Sheppard, IRL Press at Oxford University, 1989, ISBN 1 85221 134 2 and ISBN 1 85221 133 4 and more recently in monographs such as “Fmoc Solid Phase Peptide Synthesis—A Practical Approach” by W. C. Chan and P. D. White, Oxford University Press, 2000, ISBN 0 19 963 725 3 and ISBN 0 19 963 725 4. See also U.S. Pat. No. 6,281,331.

The solid phase synthesis/filtration vessel containing the resin-bound peptide was cooled to 0 to −10° C. and dichloromethane 368 kg, 9-10 volumes, was charged to a second vessel. Trifluoroacetic acid (TFA, 8.6 kg, 0.2 volumes relative to resin weight) was added to the vessel containing the dichloromethane to give a concentration of ˜2% TFA in dichloromethane. This solution was then cooled to 0 to −10° C. with moderate agitation (final temperature −4.3° C.).

The acid solution was then transferred from the second vessel to the vessel containing the resin-bound peptide and the transfer line flushed with dichloromethane (13.5 kg, 0.38 volumes relative to resin weight). The resin was rapidly agitated in the synthesis/filtration vessel to prevent clumping and to facilitate the cleavage of the peptide from the resin. The temperature was held at ˜−10 to +2° C. for at least 30 min (Actual=30 min exposure time and 1.0° C. final temperature). The cleavage reaction was terminated by the addition of pyridine (6.7 kg, 0.24 volumes, 1.2 equiv relative to TFA) directly into the synthesis/filtration vessel. The synthesis/filtration vessel jacket was set at 10° C. and the vessel purged with an inert gas such as nitrogen. The vessel was then agitated for at least 5 min at a temperature of 5 to 15° C. (Actual values 20 min stir, final temperature 7.5° C.). The cleavage solution was transferred to a third vessel for distillation.

A dichloromethane wash (187 kg, 5 to 6 volumes) was sent to the synthesis/filtration vessel and the mixture agitated for 30 min at room temperature (17 to 19°) then sent to the third distillation vessel. Concentration of the combined cleavage solution and dichloromethane wash by vacuum distillation was initiated at this point while maintaining the vessel temperature at 10 to 20° C. and the jacket temperature below 40° C. Vacuum was varied to maintain these parameters.

A second dichloromethane wash (187 kg, 5 to 6 volumes) was sent to the synthesis/filtration vessel and the mixture agitated for 30 min at room temperature then sent to the third distillation vessel. Concentration of the combined cleavage solution and dichloromethane wash by vacuum distillation was continued at this point while maintaining the vessel temperature at 10 to 20° C. and the jacket temperature below 40° C. Vacuum was varied to maintain these parameters.

Ethanol containing 5% isopropanol (330 kg, 450 L) was stored in the second vessel at room temperature. The first ethanol wash of ˜120 L was sent to the synthesis/filtration vessel where the mixture was agitated for at least 15 min and then sent to holding tank.

A third dichloromethane wash (187 kg, 5 to 6 volumes) was sent to the synthesis/filtration vessel and the mixture agitated for 30 min at room temperature then sent to the third distillation vessel. Concentration of the combined cleavage solution and dichloromethane wash by vacuum distillation was continued at this point while maintaining the vessel temperature at 10 to 20° C. and the jacket temperature below 40° C. Vacuum was varied to maintain these parameters.

Concentration of the cleavage solution and dichloromethane was continued using vacuum distillation keeping the vessel temperature 10 to 20° C. and the jacket temperature below 40° C. Vacuum was varied to maintain these parameters.

Vacuum distillation was continued until a specified volume is reached (typically ˜25% of the original cleavage solution volume).

Two further ethanol washes, ˜120 to 150 L were sent to the synthesis/filtration vessel where the mixture is agitated for at least 15 min and then the washes were sequentially sent to holding tank. The combined ethanol washes were then sent to the distillation vessel and distillation continued under vacuum until a dichloromethane in ethanol concentration of 3 to 10% is attained (Actual 7.4% DCM in ethanol).

Residual non-cleaved fragment left on resin at cleavage completion was 1.47 g/kg (35 g) or 0.1% of the weight of isolated material. At the end of the wash cycle the amount of cleaved peptide which could be washed from the resin was 8.2 g/kg (200 g) or 0.5% of the isolated peptide.

Water (144 L, 5 to 6 volumes) was slowly charged to the distillation vessel with moderate to rapid agitation over 30 min maintaining a temperature of 9 to 15° C. during the addition. Using moderate to slow agitation the precipitating peptide slurry was cooled to −5 to 5° C. over at least 1 hour (Actual=4.5 h).

The precipitated peptide was then isolated in an agitated filter vessel, pre-cooled to −5 to 5° C., agitated with 1 to 3 volumes of water and filtered. It was then further agitated with −5 to 10 volumes of ethanol/water mixture (80:20) which had been pre-cooled to −5 to 5° C. and filtered. The product was blown dry with an inert gas such as nitrogen and either dried in the filter or removed from the filter and dried in an appropriate drying apparatus. Drying was continued at <100 mmHg and a jacket temperature of <45° C. until the water content of the peptide was <1%.

A yield of 37.4 kg (80.6% yield) was obtained. Theoretical yield was 46.4 kg. Purity was 96.4% by area normalized HPLC.

### EXAMPLE 4

### Cleavage and Isolation of Fmoc(17-26)OH from Its Polymeric Support (Swell/Shrink/Swell/Shrink/Swell and Salt Removal)

Fragment 2 (Fmoc(17-26)OH, Fully protected LELLEQENKE) was assembled on a 2-chlorotrityl chloride functionalized bead composed on polystyrene (PS), crosslinked with 1% divinyl benzene and preloaded with the first amino acid, Fmoc-LeuOH (24 kg loaded resin, leucine loading 1.04 mmol/g).

The removal of the Fmoc protecting group, coupling reactions and other aspects of Fmoc/t-Butyl solid phase synthesis are consistent with general methodologies known to one skilled in the art and embodied in such publications as “Solid Phase Peptide Synthesis—A Practical Approach” by E. Atherton and R. C. Sheppard, IRL Press at Oxford University, 1989, ISBN 1 85221 134 2 and ISBN 1 85221 133 4 and more recently in monographs such as “Fmoc Solid Phase Peptide Synthesis—A Practical Approach” by W. C. Chan and P. D. White, Oxford University Press, 2000, ISBN 0 19 963 725 3 and ISBN 0 19 963 725 4. See also U.S. Pat. No. 6,281,331.

The solid phase synthesis/filtration vessel containing the resin-bound peptide was cooled to 0 to −10° C. and dichloromethane 368 kg, 9-10 volumes, was charged to a second vessel. Trifluoroacetic acid (TFA, 8.5 kg, 0.2 volumes relative to resin weight) was added to the vessel containing the dichloromethane to give a concentration of ˜2% TFA in dichloromethane. This solution was then cooled to 0 to −10° C. with moderate agitation (final temperature −5.4° C.).

The acid solution was then transferred from the second vessel to the vessel containing the resin-bound peptide and the transfer line flushed with dichloromethane (13.5 kg, 0.38 volumes relative to resin weight). The resin was rapidly agitated in the synthesis/filtration vessel to prevent clumping and facilitate cleavage of the peptide from the resin. The temperature was held at ˜−10 to +2° C. for at least 30 min (Actual=60 min exposure time and 0.2° C. final temperature). The cleavage reaction was terminated by the addition of pyridine (6.7 kg, 0.24 volumes, 1.2 equiv relative to TFA) directly into the synthesis/filtration vessel. The synthesis/filtration vessel jacket was set at 10° C. and the vessel purged with an inert gas such as nitrogen. The vessel was then agitated for at least 5 min at a temperature of 5 to 15° C. (Actual values 5 min stir, final temperature 6° C.). The cleavage solution was transferred to a third vessel for distillation.

A dichloromethane wash (187 kg, 5 to 6 volumes) was sent to the synthesis/filtration vessel and the mixture agitated for 30 min at room temperature (17 to 19° C.) then sent to the third distillation vessel. Concentration of the combined cleavage solution and dichloromethane wash by vacuum distillation was initiated at this point while maintaining the vessel temperature at 10 to 20° C. and the jacket temperature below 40° C. Vacuum was varied to maintain these parameters.

A second dichloromethane wash (187 kg, 5 to 6 volumes) was sent to the synthesis/filtration vessel and the mixture agitated for 30 min at room temperature then sent to the third distillation vessel. Concentration of the combined cleavage solution and dichloromethane wash by vacuum distillation was initiated at this point while maintaining the vessel temperature at 10 to 20° C. and the jacket temperature below 40° C. Vacuum was varied to maintain these parameters.

Ethanol containing 5% isopropanol (374 kg, 513 L) was stored in the second vessel at room temperature. The first ethanol wash ˜120 L was sent to the synthesis/filtration vessel where the mixture was agitated for at least 15 min and then sent to holding tank.

A third dichloromethane wash (187 kg, 5 to 6 volumes) was sent to the synthesis/filtration vessel and the mixture agitated for 30 min at room temperature then sent to the third distillation vessel. Concentration of the combined cleavage solution and dichloromethane washes by vacuum distillation was initiated at this point while maintaining the vessel temperature at 10 to 20° C. and the jacket temperature below 40° C. Vacuum was varied to maintain these parameters.

The combined concentrated cleavage solution and dichloromethane washes are washed with water (2×90 kg, 2×4 volumes) following addition of the third wash to the distillation vessel to remove TFA salts. The aqueous layers are discarded and distillation continued.

Concentration of the cleavage solution and dichloromethane was continued using vacuum distillation keeping the vessel temperature 10 to 20° C. and the jacket temperature below 40° C. Vacuum was varied to maintain these parameters.

Vacuum distillation was continued until a specified volume is reached (typically ˜25% of the original cleavage solution volume).

Two further ethanol washes ˜120 to 150 L were sent to the synthesis/filtration vessel where the mixture is agitated for at least 15 min and then the washes are sequentially sent to holding tank. The combined ethanol washes were then sent to the distillation vessel and distillation continued under vacuum until a dichloromethane in ethanol concentration of 3 to 10% is attained (Actual 7.4% DCM in ethanol).

Residual non-cleaved fragment left on resin at cleavage completion was 0.97 g/kg (23 g) or 0.05% of the weight of isolated material. At the end of the wash cycle the amount of cleaved peptide which could be washed from the resin was 1.67 g/kg (40 g) or 0.09% of the isolated peptide.

Water (144 L, 5 to 6 volumes) was slowly charged to the distillation vessel with moderate to rapid agitation over 30 min maintaining a temperature of 9 to 15° C. during the addition. Using moderate to slow agitation the precipitating peptide slurry is cooled to −5 to 5° C. over at least 1 hour (Actual=1.0 h).

The precipitated peptide was then isolated in a self discharging centrifuge, pre-cooled to −5 to 5° C., washed with 1 to 3 volumes of water, then washed with 5 to 10 volumes of ethanol/water mixture (80:20) which had been pre-cooled to −5 to 5° C. The product was blown dry with an inert gas such as nitrogen and either dried in the filter or removed from the filter and dried in an appropriate drying apparatus. Drying was continued at <100 mmHg and a jacket temperature of <45° C. until the water content of the peptide was <1%.

A yield of 45.9 kg (80.7% yield) was obtained. Theoretical yield was 56.9 kg. Purity was 94.1% by area normalized HPLC.

The data obtained in Examples 1-4 demonstrate that yield improved 5 to 6% by using the methodology of the present invention. Purity was slightly lowered but still within acceptable specifications. Without repeating the swell/shrink cycle, the yield was only about 74.5%. When practicing the invention, the yield improved to about 80%. This improvement is believed to be due to mechanical extrusion of hard to access peptide from the bead interior results in higher extraction efficiencies.

### EXAMPLE 5

### Cleavage and Isolation of Fmoc(27-35)OH from Its Polymeric Support (Shrink/Swell Only)

Fragment 3 (Fmoc(27-35)OH refers to a globally protected peptide according to Seq ID No. 17 in which the N-terminus bears an FMOC protecting group, side group protecting groups, and no protecting group at the C-terminus after being cleaved from the resin support (i.e., fully protected FmocLELLEQENKE). Fragment 3 (Fmoc(27-35)OH, fully protected FmocDKWASLWNW) was assembled on a 2-chlorotrityl chloride functionalized bead composed on polystyrene (PS), crosslinked with 1% divinyl benzene and preloaded with the first amino acid, Fmoc-Trp(Boc)OH (15 kg loaded resin, Tryptophan loading 0.60 mmol/g).

The removal of the Fmoc protecting group, coupling reactions and other aspects of Fmoc/t-Butyl solid phase synthesis are consistent with general methodologies known to one skilled in the art and embodied in such publications as “Solid Phase Peptide Synthesis—A Practical Approach” by E. Atherton and R. C. Sheppard, IRL Press at Oxford University, 1989, ISBN 1 85221 134 2 and ISBN 1 85221 133 4 and more recently in monographs such as “Fmoc Solid Phase Peptide Synthesis—A Practical Approach” by W. C. Chan and P. D. White, Oxford University Press, 2000, ISBN 0 19 963 725 3 and ISBN 0 19 963 725 4. See also U.S. Pat. No. 6,281,331.

The solid phase synthesis/filtration vessel, containing the resin-bound peptide, was cooled to 0 to −10° C. and dichloromethane 195 kg, 9-10 volumes, was charged to a second vessel. Trifluoroacetic acid (TFA, 4.4 kg, 0.2 volumes relative to resin weight) was added to the vessel containing the dichloromethane to give a concentration of ˜2% TFA in dichloromethane. This solution was then cooled to 0 to −10° C. with moderate agitation (final temperature −2.0° C.).

The acid solution was then transferred from the second vessel to the synthesis vessel containing the resin-bound peptide and the transfer line flushed with dichloromethane (10 kg, 0.50 volumes relative to resin weight). The resin was rapidly agitated to prevent clumping in the synthesis/filtration vessel and to facilitate cleavage of the peptide from the resin. The temperature was held at −10 to +2° C. for at least 30 min (Actual=30 min exposure time and −0.1° C. final temperature). The cleavage reaction was terminated by the addition of pyridine (3.5 kg, 0.24 volumes, 1.2 equiv relative to TFA) directly into the synthesis/filtration vessel. The synthesis/filtration vessel jacket is set at 10° C. and the vessel purged with an inert gas such as nitrogen. The vessel was then agitated for at least 5 min at a temperature of 5 to 15° C. (Actual values 5 min stir, final temperature 7° C.). The cleavage solution was transferred to a third vessel for distillation.

A dichloromethane rinse (60 kg) was sent to the second vessel and then sent to the synthesis/filtration vessel and straight to the distillation vessel after agitating for 5 min.

Ethanol (195 kg, 260 L) was stored in the second vessel at room temperature. The first ethanol wash of ˜100 L was sent to the synthesis/filtration vessel and left unagitated. This ethanol wash was then sent to the distillation vessel. Concentration of the combined cleavage solution and wash by vacuum distillation was initiated at this point while maintaining the vessel temperature at 10 to 20° C. and the vessel jacket temperature below 40° C. Vacuum was varied to maintain these parameters.

Two further ethanol washes of 90 L and 70 L were sent to the synthesis/filtration vessel where the mixture was held for at least 5 min and then the washes were sent to the distillation vessel as volume allowed. The combined DCM and ethanol washes were subjected to distillation which was continued under vacuum until a dichloromethane in ethanol concentration of 3 to 8% is attained (Actual 4.1% DCM in ethanol).

At the end of the wash cycle the cleaved peptide still washing out the resin was not quantified.

Water (75 L, 5 volumes) was slowly charged to the distillation vessel with moderate to rapid agitation over 30 min while maintaining a temperature of 9 to 15° C. during the addition. Using moderate to slow agitation the precipitating peptide slurry was cooled to −5 to 5° C. over at least 1 hour (Actual=1.1 h, final temp 1° C.).

The precipitated peptide was then isolated in an agitated filter vessel, pre-cooled to −5 to 5° C., agitated with 2 volumes of water and filtered, then agitated with 5 to 10 volumes of ethanol/water mixture (90:10) which had been pre-cooled to −5 to 5° C. and filtered. The product was blown dry with an inert gas such as nitrogen and either dried in the filter or removed from the filter and dried in an appropriate drying apparatus. Drying was continued at <100 mmHg and a jacket temperature of <45° C. until the water content of the peptide was <1%.

A yield of 14.7 kg (75.0% Theory) was obtained. Theoretical yield was 19.6 kg. Purity was 92.4% by area normalized HPLC.

### EXAMPLE 6

### Cleavage and Isolation of Fmoc(27-35)OH from Its Polymeric Support (Swell/Shrink Only)

Fragment 3 (Fmoc(27-35)OH, Fully protected FmocDKWASLWNW) was assembled on a 2-chlorotrityl chloride functionalized bead composed on polystyrene (PS), crosslinked with 1% divinyl benzene and preloaded with the first amino acid, Fmoc-Trp(Boc)OH (18 kg loaded resin, Tryptophan loading 0.61 mmol/g).

The removal of the Fmoc protecting group, coupling reactions and other aspects of Fmoc/t-Butyl solid phase synthesis are consistent with general methodologies known to one skilled in the art and embodied in such publications as “Solid Phase Peptide Synthesis—A Practical Approach” by E. Atherton and R. C. Sheppard, IRL Press at Oxford University, 1989, ISBN 1 85221 134 2 and ISBN 1 85221 133 4 and more recently in monographs such as “Fmoc Solid Phase Peptide Synthesis—A Practical Approach” by W. C. Chan and P. D. White, Oxford University Press, 2000, ISBN 0 19 963 725 3 and ISBN 0 19 963 725 4. See also U.S. Pat. No. 6,281,331.

The solid phase synthesis/filtration vessel, containing the resin-bound peptide, was cooled to 0 to −10° C. and dichloromethane 234 kg, 9-10 volumes, was charged to a second vessel. Trifluoroacetic acid (TFA, 5.4 kg, 0.2 volumes relative to resin weight) was added to the vessel containing the dichloromethane to give a concentration of 2% TFA in dichloromethane. This solution was then cooled to 0 to −10° C. with moderate agitation (final temperature −2.0° C.).

The acid solution was then transferred from the second vessel to the synthesis vessel containing the resin-bound peptide and the transfer line flushed with dichloromethane (7.2 kg, 0.30 volumes relative to resin weight). The resin was rapidly agitated to prevent clumping in the synthesis/filtration vessel and to facilitate cleavage of the peptide from the resin. The temperature was held at −10 to +2° C. for at least 30 min (Actual=30 min exposure time and −0.5° C. final temperature). The cleavage reaction was terminated by the addition of pyridine (4.2 kg, 0.24 volumes, 1.2 equiv relative to TFA) directly into the synthesis/filtration vessel. The synthesis/filtration vessel jacket is set at 10° C. and the vessel purged with an inert gas such as nitrogen. The vessel was then agitated for at least 5 min at a temperature of 5 to 15° C. (Actual values 6 min stir, final temperature 6° C.). The cleavage solution may be transferred to a third vessel for distillation.

A dichloromethane wash (119.2 kg) was sent to the second vessel and then sent to the synthesis/filtration vessel and agitated for 30 min at 17 to 23° C. (Actual 30 min, 19.3° C.). This wash was sent to the distillation vessel to join the cleavage solution. At the end of the first DCM wash cycle the cleaved peptide still washing out the resin was 15.5 mg/g (280 g) or 1.46% of the total isolated peptide.

A second dichloromethane wash (119.2 kg) was sent to the second vessel and then sent to the synthesis/filtration vessel and agitated for 30 min at 17 to 23° C. (Actual 30 min, 19.3° C.). This wash was sent to the distillation vessel to join the cleavage solution. At the end of the second DCM wash it was 7.22 mg/g (130 g) or 0.7% of the total isolated peptide.

Concentration of the combined cleavage solution and wash by vacuum distillation was initiated at this point while maintaining the vessel temperature at 10 to 20° C. and the vessel jacket temperature below 40° C. Vacuum was varied to maintain these parameters.

Ethanol (163 kg, 224 L) was stored in the second vessel at room temperature. The first ethanol wash ˜100 L was sent to the synthesis/filtration vessel and agitated for at least 5 min at room temperature (Actual 30 min, 20.1° C.). This ethanol wash was then sent to the distillation vessel. Concentration of the combined cleavage solution and wash by vacuum distillation was initiated at this point while maintaining the vessel temperature at 10 to 20° C. and the vessel jacket temperature below 40° C. Vacuum was varied to maintain these parameters.

Two further ethanol washes ˜60 to 70 L a were sent to the synthesis/filtration vessel where the mixture was held for at least 5 min and then the washes were sent to the distillation vessel as volume allowed. The combined DCM and ethanol washes were subjected to distillation which was continued under vacuum until a dichloromethane in ethanol concentration of 3 to 8% is attained (Actual 7.1% DCM in ethanol).

Water (90 L, 5 volumes) was slowly charged to the distillation vessel with moderate to rapid agitation over 30 min while maintaining a temperature of 9 to 15° C. during the addition. Using moderate to slow agitation the precipitating peptide slurry was cooled to −5 to 5° C. over at least 1 hour (Actual=1.0 h, final temp 1.4° C.).

The precipitated peptide was then isolated in an agitated filter vessel, pre-cooled to −5 to 5° C., agitated with 2 volumes of water and filtered, then agitated with 5 to 10 volumes of ethanol/water mixture (90:10) which had been pre-cooled to −5 to 5° C. and filtered. The product was blown dry with an inert gas such as nitrogen and either dried in the filter or removed from the filter and dried in an appropriate drying apparatus. Drying was continued at <100 mmHg and a jacket temperature of <45° C. until the water content of the peptide was <1%.

A yield of 19.1 kg (79.5% Theory) was obtained. Theoretical yield was 24 kg. Purity was 92.4% by area normalized HPLC.

### EXAMPLE 7

### Cleavage and Isolation of Fmoc(27-35)OH from Its Polymeric Support (Swell/Shrink/Swell/Shrink/Swell)

Fragment 3 (Fmoc(27-35)OH, Fully protected FmocDKWASLWNW) was assembled on a 2-chlorotrityl chloride functionalized bead composed on polystyrene (PS), crosslinked with 1% divinyl benzene and preloaded with the first amino acid, Fmoc-Trp(Boc)OH (22.4 kg loaded resin, Tryptophan loading 0.62 mmol/g).

The removal of the Fmoc protecting group, coupling reactions and other aspects of Fmoc/t-Butyl solid phase synthesis are consistent with general methodologies known to one skilled in the art and embodied in such publications as “Solid Phase Peptide Synthesis—A Practical Approach” by E. Atherton and R. C. Sheppard, IRL Press at Oxford University, 1989, ISBN 1 85221 134 2 and ISBN 1 85221 133 4 and more recently in monographs such as “Fmoc Solid Phase Peptide Synthesis—A Practical Approach” by W. C. Chan and P. D. White, Oxford University Press, 2000, ISBN 0 19 963 725 3 and ISBN 0 19 963 725 4. See also U.S. Pat. No. 6,281,331.

The solid phase synthesis/filtration vessel, containing the resin-bound peptide, was cooled to 0 to −10° C. and dichloromethane 290 kg, 9-10 volumes, was charged to a second vessel. Trifluoroacetic acid (TFA, 6.7 kg, 0.2 volumes relative to resin weight) was added to the vessel containing the dichloromethane to give a concentration of ˜2% TFA in dichloromethane. This solution was then cooled to 0 to −10° C. with moderate agitation (final temperature −4.6° C.).

The acid solution was then transferred from the second vessel to the synthesis vessel containing the resin-bound peptide and the transfer line flushed with dichloromethane (11.0 kg, 0.30 volumes relative to resin weight). The resin was rapidly agitated to prevent clumping in the synthesis/filtration vessel and to facilitate cleavage of the peptide from the resin. The temperature was held at −10 to +2° C. for at least 30 min (Actual=30 min exposure time and 1.5° C. final temperature). The cleavage reaction was terminated by the addition of pyridine (5.2 kg, 0.24 volumes, 1.2 equiv relative to TFA) directly into the synthesis/filtration vessel. The synthesis/filtration vessel jacket is set at 10° C. and the vessel purged with an inert gas such as nitrogen. The vessel was then agitated for at least 5 min at a temperature of 5 to 15° C. (Actual values 6 min stir, final temperature 6.2° C.). The cleavage solution may be transferred to a third vessel for distillation.

A dichloromethane wash (149 kg) was sent to the second vessel and then sent to the synthesis/filtration vessel and agitated for 30 min at 17 to 23° C. (Actual 30 min, 19.3° C.). This wash was sent to the distillation vessel to join the cleavage solution. Concentration of the combined cleavage solution and wash by vacuum distillation was initiated at this point while maintaining the vessel temperature at 10 to 20° C. and the vessel jacket temperature below 40° C. Vacuum was varied to maintain these parameters.

Ethanol (290 kg, 398 L) was stored in the second vessel at room temperature. The first ethanol wash ˜120 L was sent to the synthesis/filtration vessel where the mixture is agitated for at least 15 min and then sent to holding tank.

At the end of the first DCM wash cycle the cleaved peptide still washing out the resin was 63.6 mg/g (1420 g) or 6.3% of the total isolated peptide A second dichloromethane wash (149 kg) was sent to the second vessel and then sent to the synthesis/filtration vessel and agitated for 30 min at 17 to 23° C. (Actual 30 min, 19.3° C.). This wash was sent to the distillation vessel to join the cleavage solution. At the end of this swell and shrink cycle the cleaved peptide still washing out the resin was 7.22 mg/g (130 g) or 0.7% of the total isolated peptide.

The second ethanol wash ˜120 L was sent to the synthesis/filtration vessel where the mixture is agitated for at least 15 min and then sent to holding tank.

At the end of this swell and shrink cycle the cleaved peptide still washing out the resin was 9 mg/g (200 g) or 0.9% of the total isolated peptide.

A final ethanol wash ˜150 L was sent to the synthesis/filtration vessel where the mixture was held for at least 5 min and then the washes were sent to the distillation vessel as volume allowed. The combined DCM and ethanol washes were subjected to distillation which was continued under vacuum until a dichloromethane in ethanol concentration of 3 to 8% is attained (Actual 7.3% DCM in ethanol, 15.6° C.).

Water (112 L, 5 volumes) was slowly charged to the distillation vessel with moderate to rapid agitation over 30 min while maintaining a temperature of 9 to 15° C. during the addition. Using moderate to slow agitation the precipitating peptide slurry was cooled to −5 to 5° C. over at least 1 hour (Actual=1.2 h, final temp 4.1° C.).

The precipitated peptide was then isolated in a filter press apparatus, pre-cooled to −5 to 5° C., agitated with 2 volumes of water and filtered, then agitated with 5 to 10 volumes of ethanol/water mixture (90:10) which had been pre-cooled to −5 to 5° C. and filtered. The product was blown dry with an inert gas such as nitrogen and either dried in the filter or removed from the filter and dried in an appropriate drying apparatus. Drying was continued at <100 mmHg and a jacket temperature of <45° C. until the water content of the peptide was <1%.

A yield of 24.2 kg (80% Theory) was obtained. Theoretical yield was 30.3 kg. Purity 87.3% by area normalized HPLC.

### EXAMPLE 8

### Cleavage and Isolation of Fmoc(27-35)OH from Its Polymeric Support (Swell/Shrink/Swell/Shrink and Water Washes)

Fragment 3 (Fmoc(27-35)OH, Fully protected FmocDKWASLWNW) was assembled on a 2-chlorotrityl chloride functionalized bead composed on polystyrene (PS), crosslinked with 1% divinyl benzene and preloaded with the first amino acid, Fmoc-Trp(Boc)OH (24 kg loaded resin, Tryptophan loading 0.64 mmol/g).

The removal of the Fmoc protecting group, coupling reactions and other aspects of Fmoc/t-Butyl solid phase synthesis are consistent with general methodologies known to one skilled in the art and embodied in such publications as “Solid Phase Peptide Synthesis—A Practical Approach” by E. Atherton and R. C. Sheppard, IRL Press at Oxford University, 1989, ISBN 1 85221 134 2 and ISBN 1 85221 133 4 and more recently in monographs such as “Fmoc Solid Phase Peptide Synthesis—A Practical Approach” by W. C. Chan and P. D. White, Oxford University Press, 2000, ISBN 0 19 963 725 3 and ISBN 0 19 963 725 4. See also U.S. Pat. No. 6,281,331.

The solid phase synthesis/filtration vessel, containing the resin-bound peptide, was cooled to 0 to −10° C. and dichloromethane 320 kg, 9-10 volumes, was charged to a second vessel. Trifluoroacetic acid (TFA, 8.5 kg, 0.2 volumes relative to resin weight) was added to the vessel containing the dichloromethane to give a concentration of 2% TFA in dichloromethane. This solution was then cooled to 0 to −10° C. with moderate agitation (final temperature −1.6° C.).

The acid solution was then transferred from the second vessel to the synthesis vessel containing the resin-bound peptide and the transfer line flushed with dichloromethane (14.2 kg, 0.30 volumes relative to resin weight). The resin was rapidly agitated to prevent clumping in the synthesis/filtration vessel and to facilitate cleavage of the peptide from the resin. The temperature was held at −10 to +2° C. for at least 30 min (Actual=30 min exposure time and −0.7° C. final temperature). The cleavage reaction was terminated by the addition of pyridine (6.7 kg, 0.24 volumes, 1.2 equiv relative to TFA) directly into the synthesis/filtration vessel. The synthesis/filtration vessel jacket is set at 10° C. and the vessel purged with an inert gas such as nitrogen. The vessel was then agitated for at least 5 min at a temperature of 5 to 15° C. (Actual values 5 min stir, final temperature 6.1° C.). The cleavage solution may be transferred to a third vessel for distillation. After cleavage only 62 g of non cleaved product was left on the resin.

A dichloromethane wash (191 kg) was sent to the second vessel and then sent to the synthesis/filtration vessel and agitated for 30 min at 17 to 23° C. This wash was sent to the distillation vessel to join the cleavage solution. This process was repeated with a second 191 kg dichloromethane wash. Concentration of the combined cleavage solution and wash by vacuum distillation was initiated at this point while maintaining the vessel temperature at 10 to 20° C. and the vessel jacket temperature below 40° C. Vacuum was varied to maintain these parameters.

Ethanol (280 kg, 385 L) was stored in the second vessel at room temperature. The first ethanol wash ˜120 L was sent to the synthesis/filtration vessel where the mixture is agitated for at least 15 min and then sent to holding tank.

At the end of the first ethanol wash the cleaved peptide still washing out the resin was 55.7 mg/g (1.34 kg) or 4.9% of the total isolated peptide.

A third dichloromethane wash (191 kg) was sent to the second vessel and then sent to the synthesis/filtration vessel and agitated for 30 min at 17 to 23° C. This wash was sent to the distillation vessel to join the cleavage solution. The concentrated cleavage solution and dichloromethane washes were then extracted twice with 90 L of water to remove residual TFA salts. The aqueous layer was discarded.

The second ethanol wash ˜120 L was sent to the synthesis/filtration vessel where the mixture is agitated for at least 15 min and then sent to holding tank. At the end of the second swell and shrink cycle the cleaved peptide still washing out the resin was 14.3 mg/g (0.34 kg) or 1.2% of the total isolated peptide.

A fourth dichloromethane wash (191 kg) was sent to the second vessel and then sent to the synthesis/filtration vessel and agitated for 30 min at 17 to 23° C. This wash was sent to the distillation vessel to join the cleavage solution.

The remaining ethanol wash was sent to the synthesis/filtration vessel where the mixture is agitated for at least 15 min and then sent to holding tank. At the end of this swell and shrink cycle the cleaved peptide still washing out the resin was 4 mg/g (96 g) or 0.3% of the total isolated peptide.

The combined DCM and ethanol washes were subjected to distillation which was continued under vacuum until a dichloromethane in ethanol concentration of 3 to 8% is attained (Actual 6.6% DCM in ethanol, 16.2° C.).

Water (144 L, 5 volumes) was slowly charged to the distillation vessel with moderate to rapid agitation over 30 min while maintaining a temperature of 9 to 15° C. during the addition. Using moderate to slow agitation the precipitating peptide slurry was cooled to −5 to 5° C. over at least 1 hour (Actual=1 h, final temp 2.0° C.).

The precipitated peptide was then isolated in a self discharging centrifuge, pre-cooled to −5 to 5° C., agitated with 2 volumes of water and filtered, then agitated with 5 to 10 volumes of ethanol/water mixture (90:10) which had been pre-cooled to −5 to 5° C. and filtered. The product was blown dry with an inert gas such as nitrogen and either dried in the filter or removed from the filter and dried in an appropriate drying apparatus. Drying was continued at <100 mmHg and a jacket temperature of <45° C. until the water content of the peptide was <1%.

A yield of 27.5 kg (82.1% Theory) was obtained. Theoretical yield was 33.5 kg. Purity was 90.6% by area normalized HPLC.

### EXAMPLE 9

### Repeating the Procedure of Example 8 at Pilot Plant and Production Scales

The procedures of Examples 7 and 8 were generally repeated at pilot plant and/or production scales. As shown by the following data, the practice of the present invention provides a 3% yield improvement at pilot plant scale and about a 6% yield improvement at full production scale. The improved performance at larger scales is a significant accomplishment. This is believed to be due to mechanical extrusion of hard to access peptide from the bead interior results in higher extraction efficiencies. Purity was slightly lowered but still within expectations.

TABLE 9A
Pilot Scale Comparison Data

| | No. | | Average |
|---|---|---|---|
| | Batches | % Yield | Purity |
| Process | Completed | (Average) | (%) |
| Original Process | 6 | 80.7 | 91.3 |
| Swell/Shrink/Swell/Shrink | 1 | 79.8 | 87.3 |
| Swell/Shrink/Swell/Shrink and water | 4 | 83.8 | 89.7 |
| wash | | | |

TABLE 9B
Full Production Scale Data for Swell/Shrink/Swell/Shrink and
water wash process (Example 8)

| | No. | | Average |
|---|---|---|---|
| | Batches | % Yield | Purity |
| Process | Completed | (Average) | (%) |
| Process Validation Batches | 4 | 80.1 | 87.5 |
| | | (range 76 to 86) | |
| Post Validation Batches | 2 | 86.7 | 88.4 |

Other embodiments of this invention will be apparent to those skilled in the art upon consideration of this specification or from practice of the invention disclosed herein. Various omissions, modifications, and changes to the principles and embodiments described herein may be made by one skilled in the art without departing from the true scope and spirit of the invention which is indicated by the following claims.

All patents, patent documents, and publications cited herein are hereby incorporated by reference as if individually incorporated.

1. A method of obtaining a cleaved peptide from a support, comprising the steps of:
a) providing a composition comprising a cleaved peptide in admixture with a support from which the peptide has been cleaved; and
b) subjecting the composition to an alternating and at least partially repeating cycle of swelling and shrinking treatments during which the peptide is extracted into one or more washes.

2. The method of claim 1, wherein the cleaved peptide comprises a peptide intermediate fragment of enfuvirtide.

3. The method of claim 2, wherein the cleaved peptide comprises protecting groups.

4. The method of claim 2, wherein the cleaved peptide is at least substantially globally protected.

5. The method of claim 2, wherein the cleaved peptide comprises an amino acid sequence according to Seq. Id. No. 11.

6. The method of claim 2, wherein the cleaved peptide comprises an amino acid sequence according to Seq. Id. No. 17.

7. The method of claim 1, wherein a swelling treatment comprises contacting the composition with a liquid wash comprising a chlorinated solvent.

8. The method of claim 7, wherein the chlorinated solvent is dichloromethane.

9. The method of claim 1, wherein a shrinking treatment comprises contacting the composition with a liquid wash comprising an alcohol.

10. The method of claim 1, further comprising recovering the peptide from a mixture comprising shrinking and swelling washes.

11. The method of claim 10, wherein said recovering step comprises adding a precipitating liquid to the concentrated mixture.

12. The method of claim 11, wherein, prior to adding a precipitating liquid, concentrating the mixture while information indicative of a relative amount of a liquid in the mixture is monitored and stopping concentrating of the mixture in response to information indicating that the concentrated mixture comprises a desired relative amount of the liquid in the mixture.

13. The method of claim 9, wherein the alcohol comprises ethanol.

14. The method of claim 9, wherein the alcohol comprises isopropyl alcohol.

15. A method of obtaining a cleaved peptide from a support, comprising the steps of:
providing a composition comprising a cleaved peptide in admixture with a support from which the peptide has been cleaved;
contacting the composition with one or more swelling washes under conditions such that at least a portion of the peptide is extracted into the one or more swelling washes;
contacting the composition with one or more shrinking washes;
contacting the composition with one or more additional swelling washes under conditions such that at least a portion of the peptide is extracted into the one or more additional swelling washes; and
recovering the peptide from one or more of the swelling washes.

16. The method of claim 15, further comprising recovering peptide from one or more of the shrinking washes.

17. A method of obtaining a peptide from a polymeric support resin, comprising the steps of:
providing a composition comprising a globally protected peptide attached to a support resin;
cleaving the peptide from the resin under conditions such that the cleaved peptide is at least substantially globally protected;
subjecting the resin and cleaved, protected peptide to a cycle of alternating and at least partially repeating cycle of swelling and shrinking treatments, wherein each swelling treatment comprises one or more swelling washes into which the peptide is extracted and each shrinking treatment comprises one or more shrinking washes into which the peptide is extracted;
concentrating the swelling and shrinking washes;
causing the peptide in the concentrated washes to precipitate; and
recovering the precipitated peptide.

18. The method of claim 14, further comprising the step of washing at least one of the swelling washes with an aqueous wash.


SEQUENCE LISTING (19 sequences)

SEQ ID NO: 1 - protein, 36 aa, Human immunodeficiency virus
YTSLIHSLIEESQNQQEKNEQELLELDKWASLWNWF

SEQ ID NO: 2 - protein, 8 aa, Artificial Sequence
  Chemically synthesized peptide fragment
YTSLIHSL

SEQ ID NO: 3 - protein, 15 aa, Artificial Sequence
  Chemically synthesized peptide fragment
YTSLIHSLIEESQNQ

SEQ ID NO: 4 - protein, 16 aa, Artificial Sequence
  Chemically synthesized peptide fragment
YTSLIHSLIEESQNQQ

SEQ ID NO: 5 - protein, 18 aa, Artificial Sequence
  Chemically synthesized peptide fragment
YTSLIHSLIEESQNQQEK

SEQ ID NO: 6 - protein, 7 aa, Artificial Sequence
  Chemically synthesized peptide fragment
IEESQNQ

SEQ ID NO: 7 - protein, 8 aa, Artificial Sequence
  Chemically synthesized peptide fragment
IEESQNQQ

SEQ ID NO: 8 - protein, 20 aa, Artificial Sequence
  Chemically synthesized peptide fragment
QEKNEQELLELDKWASLWNW

SEQ ID NO: 9 - protein, 21 aa, Artificial Sequence
  Chemically synthesized peptide fragment
QEKNEQELLELDKWASLWNWF

SEQ ID NO: 10 - protein, 7 aa, Artificial Sequence
  Chemically synthesized peptide fragment
EKNEQEL

SEQ ID NO: 11 - protein, 10 aa, Artificial Sequence
  Chemically synthesized peptide fragment
EKNEQELLEL

SEQ ID NO: 12 - protein, 20 aa, Artificial Sequence
  Chemically synthesized peptide fragment
EKNEQELLELDKWASLWNWF

SEQ ID NO: 13 - protein, 17 aa, Artificial Sequence
  Chemically synthesized peptide fragment
NEQELLELDKWASLWNW

SEQ ID NO: 14 - protein, 18 aa, Artificial Sequence
  Chemically synthesized peptide fragment
NEQELLELDKWASLWNWF

SEQ ID NO: 15 - protein, 12 aa, Artificial Sequence
  Chemically synthesized peptide fragment
LELDKWASLWNW

SEQ ID NO: 16 - protein, 13 aa, Artificial Sequence
  Chemically synthesized peptide fragment
LELDKWASLWNWF

SEQ ID NO: 17 - protein, 9 aa, Artificial Sequence
  Chemically synthesized peptide fragment
DKWASLWNW

SEQ ID NO: 18 - protein, 10 aa, Artificial Sequence
  Chemically synthesized peptide fragment
DKWASLWNWF

SEQ ID NO: 19 - protein, 19 aa, Artificial Sequence
  Chemically synthesized peptide fragment
EKNEQELLELDKWASLWNW